(19) United States
(12) Patent Application Publication
(10) Pub. No.: US 2022/0226204 A1
(43) Pub. Date: Jul. 21, 2022

(54) IMPROVEMENTS IN RELATION TO PERSONAL CLEANSING COMPOSITIONS

(71) Applicant: THOS. BENTLEY & SON LIMITED, Leeds (GB)

(72) Inventors: John Michael STORY, Leeds (GB); Charlotte GRONAN, Leeds (GB); Ravi RATHOD, Leeds (GB)

(21) Appl. No.: 17/614,536

(22) PCT Filed: May 29, 2020
(86) PCT No.: PCT/GB2020/051316

§ 371 Date: Nov. 28, 2021

(30) Foreign Application Priority Data
May 30, 2019 (GB) 1907662.9

(51) Int. Cl. A61K 8/02; A61Q 19/10; A61K 8/34; A61K 8/44; A61K 8/92; A61K 8/46; A61K 8/86; A61K 8/368; A61K 8/37

(52) U.S. Cl. CPC A61K 8/0216; A61Q 19/10; A61K 8/345; A61K 8/44; A61K 8/37; A61K 8/466; A61K 8/86; A61K 8/368; A61K 8/342; A61K 8/922

(57) ABSTRACT

The invention relates to solid personal cleansing compositions which comprise a non-soap anionic surfactant, a carrier, an emulsifier, a humectant and water. The method entails preparing an emulsion of the components at an elevated temperature and cooling the emulsion to produce a block which can be re-melted and additised with colour, fragrance by downstream processors. The invention further relates to the emulsions and to methods for preparing the cleansing compositions.

This invention relates to improvements in relation to personal cleansing compositions in solid form, for example in block, bar or tablet form. In addition to the personal cleansing compositions themselves, the invention relates to their manufacture, to emulsions from which they may be produced, and to the use of such cleansing compositions in the production of individual personal cleansing bars or tablets.

Soap (that is, fatty acid salts) is an inexpensive solid personal cleansing composition. The large scale manufacture of soap is efficient. However soap is not a product which is universally favoured by consumers. The pH of most soaps is about 9-10, whereas the natural pH of skin is about 5.5. Soap is an effective cleanser but is widely considered to be detrimental to skin. For this reason many consumers avoid soap and instead use “cleansing bars” which contain no soap or only a low proportion of soap.

Many cleansing bars (also called “detergent bars” or “syndet bars”) contain an anionic surfactant as a major component. The anionic surfactant may typically be a sulfate, sulfosuccinate or isethionate surfactant. However for reasons associated with the cost of ingredients, or for reasons associated with the manufacture, or both, cleansing bars are typically significantly more expensive than soap bars.

GB 783027 describes a detergent tablet which contains an alkali metal acyl isethionate and further components, which may include up to 25% of alkali metal soap. It is known in the art that care must be taken to avoid reaction between the alkali metal acyl isethionate and the soap. It is stated in the examples that the ingredients are mixed to form a hot pasty mass which can then be milled and plodded. It may be inferred that the alkali metal acyl isethionate and the soap do not react in the hot pasty mass.

WO 99/10467 describes a detergent formulation made by spray drying a dispersion or emulsion of very high water content, comprising a synthetic detergent and a hydrophobic plasticiser, to produce a particulate product of very low water content.

It is an object of embodiments of the present invention to provide a solid cleansing composition which is simple to manufacture and which may be melted and re-cast to form individual personal cleansing bars by customers, without degradation of properties of the composition.

It has proved exceeding difficult to produce a so-called ‘melt and pour’ solid cleansing composition. Nevertheless it is an object of embodiments of the present invention to provide a cleansing composition in solid form which may be re-melted by producers of individual personal cleansing bars and cast to form high-quality products.

In accordance with a first aspect of the present invention there is provided a solid cleansing composition comprising:
(a) at least 12% wt of a non-soap anionic surfactant having a melting point of at least 45° C.;
(b) at least 8% wt of a carrier having a melting point of at least 45° C.;
(c) at least 3% wt of an emulsifier;
(d) at least 8% wt of a humectant; and
(e) at least 8% wt water;
   wherein the composition is a solid throughout the range from 0° C. to 40° C.

Solid cleansing compositions in accordance with the first aspect of the invention may be heated, to liquefy them. The resulting heated liquid products may be cast into moulds and allowed to cool and solidify, forming individual personal cleansing bars. The personal cleansing bars have the same properties as the cleansing compositions which were melted. No discoloration, shrinkage or cracking is seen. It is believed that this highly favourable outcome, which has proved elusive until now, is related to the selection of the non-soap anionic surfactant and carrier which melt at a moderate elevated temperature; and the “support system” of emulsifier, humectant and water, all at moderate levels.

In accordance with a second aspect of the present invention there is provided a solid cleansing composition comprising:
(a) at least 15% wt and up to 40% wt of a non-soap anionic surfactant having a melting point of at least 45° C.;
(b) at least 10% wt and up to 32% wt of a carrier having a melting point of at least 45° C.;
(c) at least 4% wt and up to 14% wt of an emulsifier;
(d) at least 12% wt and up to 40% wt of a humectant;
(e) at least 10% wt and up to 36% wt of water;
   wherein components (a) to (e) make up at least 70% of the weight of the solid cleansing composition.

Suitably the cleansing composition of the first or second aspect is provided in the form of a block or tablet at ambient temperature, suitably of weight from 0.5 to 50 kg, preferably from 0.8 to 20 kg. Customers melt such blocks, or portions thereof, to form the liquid composition which is cast into individual personal cleansing bars.

Suitably the solid cleansing composition of the first or second aspect has a completely or predominantly homogeneous microstructure.

In accordance with a third aspect of the present invention there is provided an emulsion for the preparation of a solid cleansing composition, the emulsion comprising
(a) at least 11% wt of a non-soap anionic surfactant having a melting point of at least 45° C.;
(b) at least 7% wt of a carrier having a melting point of at least 45° C.;
(c) at least 3% wt of an emulsifier;
(d) at least 7 wt % of a humectant; and
(e) at least 10% wt water;
   wherein the emulsion is flowable at a temperature of at least 45° C.

In accordance with a fourth aspect of the present invention there is provided an emulsion for the preparation of a solid cleansing composition, the emulsion comprising
(a) at least 14% wt and up to 36% wt of a non-soap anionic surfactant having a melting point of at least 45° C.;
(b) at least 10% wt and up to 32% wt of a carrier having a melting point of at least 45° C.;
(c) at least 4% wt and up to 14% wt of an emulsifier;
(d) at least 10 wt % and up to 36% wt of a humectant; and
(e) and at least 12% wt and up to 40% wt water;
   wherein the emulsion is flowable at a temperature of at least 45° C., and wherein components (a) to (e) make up at least 70% of the weight of the emulsion.

In accordance with a fifth aspect of the present invention there is provided a method of making a solid cleansing product of the first or second aspect from an emulsion of the third or fourth aspect, the method comprising:
(i) providing the emulsion at a temperature at which it is flowable, and
(ii) subjecting the emulsion to cooling and solidification to form the solid cleansing product;
   wherein the method takes place without water loss or with water loss not exceeding 50% of the mass of water added to form the emulsion.

In accordance with a sixth aspect of the present invention there is provided a method of providing individual personal cleansing bars by re-melting a larger solid cleansing product of the first or second aspect, optionally adding any colorants, perfumes that are required, and casting the flowable composition into moulds. Suitably the solid cleansing product of the first or second aspect is provided in a larger size than the individual personal cleansing bars produced by the method of this sixth aspect. Therefore suitably a plurality of individual personal cleansing bars are produced from a solid cleansing product of the first or second aspect, in this method.

The following further definitions apply to all aspects of the invention, whether they be to the method, to the solid cleansing composition or to the emulsion; unless it is be stated otherwise or the context demands otherwise.

Suitably, in the third, fourth, fifth aspects the emulsion is at a temperature at which it is a sufficiently free-flowing liquid to enable it to be cast into moulds.

Suitably, in the sixth aspect the re-melted cleansing composition is at a temperature at which it is a sufficiently free-flowing liquid to enable it to be cast into moulds. Suitably the re-melted cleansing composition is solidified in said moulds to provide the individual personal cleansing bars, suitably by cooling the re-melted cleansing composition in said moulds.

In a preferred feature of any aspect of the invention the non-soap anionic surfactant (a) has a melting point of at least 50° C., the carrier (b) has a melting point of at least 50° C., and the emulsion or re-melted cleansing composition is a free-flowing liquid at a temperature of at least 50° C.

In a preferred feature of any aspect of the invention the non-soap anionic surfactant (a) has a melting point of at least 55° C., the carrier (b) has a melting point of at least 55° C., and the emulsion or re-melted cleansing composition is a free-flowing liquid at a temperature of at least 55° C.

In a preferred feature of any aspect of the invention the non-soap anionic surfactant (a) has a melting point of at least 60° C., the carrier (b) has a melting point of at least 60° C., and the emulsion or re-melted cleansing composition is a free-flowing liquid at a temperature of at least 60° C.

Suitably, in the method of the fifth aspect the emulsion preferably cools (actively or passively) and solidifies in a mould to form a block or tablet. Suitably such a block or tablet is weight from 0.5 to 50 kg, preferably from 0.8 to 20 kg.

Customers purchasing a cleansing composition of the first or second aspect can re-melt it and cast their own products. They can add colorants, perfumes, opacifiers etc as they wish. They generally produce individual personal products, typically of weight from 5 g to 300 g. Products of range 5-50 g are commonly used in the hospitality industry. Products of weight 50 g-300 g are commonly used in the home.

The cleansing compositions of this invention are suitably homogeneous in their microstructure, not heterogeneous. In preferred embodiments there is no post-solidification addition of any components to the solid cleansing composition; the entire process of making the cleansing composition preferably occurs in the liquid phase. However as noted above, producers of individual personal products who re-melt the cleansing composition have the option of adding cosmetic enhancing agents, in the liquid or solid phase.

The cleansing composition may be a facial cleanser, body cleanser, solid shampoo, cleansing shower bar or cleansing and deodorising bar.

In any aspect of the present invention it is preferred that the solid cleansing composition and/or the emulsion of the present invention do not contain any soap (salt of a fatty acid).

In any aspect of the present invention it is preferred that the solid cleansing composition and/or the emulsion of the present invention do not contain any starch.

Suitably the solid cleansing composition and/or the emulsion of the present invention does not contain any soap or starch.

Preferably in the third, fourth and fifth aspects, the emulsion is at a temperature of at least 60° C., preferably at least 70° C., preferably at least 75° C., preferably at least 80° C., preferably at least 85° C.

Preferably in the third, fourth and fifth aspects, the emulsion is at a temperature of up to 100° C., preferably up to 95° C., more preferably up to 90° C.

Preferably in the third, fourth and fifth aspects, the emulsion is at a temperature in the range 80° C. to 95° C., most preferably in the range 85° C. to 90° C.

Preferably the emulsifier (c) and humectant (d) are added to the water (e) which is at a selected elevated temperature. The surfactant (a) is added next. The carrier (b) is added to the water after the addition of the emulsifier (c), humectant (d) and the surfactant (a). The addition to the water of the emulsifier before the carrier means that the emulsifier is present to immediately start to emulsify the carrier, and preventing it from forming a film which otherwise could disrupt the process.

Other ingredients, if present, are preferably added whenever convenient. Frequently it is suitable to add them to the water with the anionic surfactant, or before.

The method of making the cleansing composition of the invention may be carried our under conditions such that substantially no water is lost in the method; that is to say, the water content in the cleansing composition is the same as in the emulsion. For example the method can be carried out in a closed environment or with return of water of evaporation. However we have found that it is not necessary to go to these lengths to operate an effective method in accordance with the invention. When the method is carried out in open environment and without capture and return of released water there is typically a modest loss of water, but a cleansing composition having good properties is still obtainable provided the other components (a), (b), (c) and (d) are present in the ranges stated above. It is believed that the presence of the humectant in the stated quantities is particularly effective in reducing/minimising water loss and therefore enabling the advantageous process for the formation of a cleansing composition of the present invention. Such loss of water is typically a passive process of water loss from the emulsion into the environment during mixing, and/or during cooling and solidification.

Whilst active cooling, for example by spraying, refrigeration or otherwise is not excluded we have found that we can achieve excellent results without it. It is not needed in effective embodiments of the invention. Thus in a preferred method of the invention the emulsion is formed at an elevated temperature and poured into a mould which is at ambient temperature, where the emulsion is allowed to cool naturally to ambient temperature, forming the solid cleansing composition.

Preferably the method takes place without water loss or with water loss not exceeding 50% of the mass of water added to form the emulsion; preferably not exceeding 40% of the mass of water added to form the emulsion; preferably not exceeding 30% of the mass of water added to form the emulsion; and most preferably not exceeding 25% of the mass of water added to form the emulsion.

It should be noted that the above definitions compare the mass of water used in the process of manufacture—that is, the mass of water in the emulsion—with the mass of water present in the solid cleansing composition. They do not define the mass of water in the emulsion expressed in terms of total mass of components in the emulsion, nor the mass of water in the solid cleansing composition expressed in terms of total mass of components in the solid cleansing composition. Such definitions are given later in this specification.

The invention provides artisan and small- and medium-scale manufacturers of cleansing products with the cleansing compositions of the invention, to which they may melt and add further components, for example colours, opacifiers and perfumes, before casting them into small moulds, to produce bars or tablets. They are used to the ‘melt and pour’ technique because of existing ‘melt and our’ soap products. The cleansing compositions of the invention are syndet products which can be melted, cosmetically modified and cast without detriment. The emulsions formed when the compositions are melted do not split. The final bars or tablets formed may have excellent aesthetics, without propensity to crack, shrink or produce a dipped top surface. Further, the resulting cleansing bars or tablets have good dissolution properties, without tendency to give any feeling of ‘grit’ (a feeling of roughness which can arise when there are differential dissolution rates of components). These excellent properties are believed to be due to the use of the defined amounts of emulsifier, humectant and water, to stabilise the defined anionic surfactant and carrier.

Suitably component (a) may comprise a sulfate, sulfonate, amphoacetate, sulfoacetate, sulfosuccinate, phosphate or carboxylate non-soap anionic surfactant; selected in each case to have a melting point of at least 45° C.

Suitably component (a) may have a melting point of at least 50° C., preferably of at least 55° C., more preferably of at least 60° C. In some preferred embodiments component (a) has a melting point of at least 65° C.

Sulfate non-soap anionic surfactants may include ammonium lauryl sulfate, sodium lauryl sulfate (SLS), fatty alcohol sulfates, and alkyl-ether sulfates, for example sodium laureth sulfate (SLES), sodium myreth sulfate and polyoxyethylene fatty alcohol ether sulfates.

Sulfonate non-soap anionic surfactants may include alkyl sulfonates, alkylbenzene sulfonates, alkenyl sulfonates, alkyl succinate sulfonates, alkylphenol sulfonates perflouoralkylsulfonates and acyl isethionates. A suitable alkyl succinate sulfonate is disodium laureth sulfosuccinate (DSLSS).

Amphoacetates, sulfoacetates, and sulfosuccinates non-soap anionic surfactants may include sodium lauryl sulfoacetate and disodium laureth sulfosuccinate.

Phosphate non-soap anionic surfactants may include alkyl-aryl ether phosphates and alkyl ether phosphates.

Carboxylate non-soap anionic surfactants may include sarcosinates, for example sodium lauroyl sarcosinate, and carboxylate-based fluorosurfactants, for example perfluorononanoate and perfluorooctanoate (PFOA or PFO).

Preferred non-soap anionic surfactants (a) for use on this invention are acyl isethionates.

An acyl isethionate for use in this invention is suitably of formula

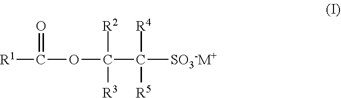

wherein R¹ is an alkyl or alkenyl group having from 8 to 24 carbon atoms;
R² is a hydrogen atom or an alkyl or alkenyl group having from 1 to 8 carbon atoms;
R³ is a hydrogen atom or an alkyl or alkenyl group having from 1 to 8 carbon atoms;
R⁴ is a hydrogen atom or an alkyl or alkenyl group having from 1 to 8 carbon atoms;
R⁵ is a hydrogen atom or an alkyl or alkenyl group having from 1 to 8 carbon atoms;
and M⁺ is a cation.

Preferably M⁺ represents an optionally substituted ammonium cation or, most preferably, a metal cation. Suitable ammonium cations include NH4⁺ and the ammonium cation of triethanolamine. Suitable metal cations include alkali metal cations, for example sodium, lithium and potassium cations, and alkaline earth metal cations, for example calcium and magnesium cations (it should be noted here that when M⁺ represents an alkaline earth metal cation M⁺ has a double positive charge and the compound has two anions). Preferably M⁺ represents a potassium cation, or, especially, a sodium cation.

R¹ may be an alkyl group or an alkenyl group. Preferably R¹ is an alkyl group. In some embodiments component (a) may comprise a surfactant derived from a mixture of fatty acids to form a mixture of compounds of formula (I) in which R¹ may be different.

R¹ is preferably the residue of a fatty acid. Fatty acids obtained from natural oils often include mixtures of fatty acids. For example the fatty acid obtained from coconut oil contains a mixture of fatty acids including C12 lauric acid, C14 myristic acid, C16 palmitic acid, C8 caprylic acid, and C18 stearic and oleic.

R¹ may include the residue of one or more naturally occurring fatty acids and/or of one or more synthetic fatty acids. In some preferred embodiments R¹ consists essentially of the residue of a single fatty acid.

Examples of carboxylic acids from which R¹ may be derived include coco acid, butyric acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from coconut oil, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof. Most preferably R¹ comprises the residue of lauric acid, that is a saturated fatty acid having 12 carbon atoms or the residue of mixed fatty acids derived from coconut oil, in which lauric acid chains predominate.

R² is preferably a hydrogen atom or an alkyl or alkenyl group having from 1 to 4 carbon atoms. Most preferably R² is a hydrogen atom.

R³ is preferably a hydrogen atom or an alkyl or alkenyl group having from 1 to 4 carbon atoms. Most preferably R³ is a hydrogen atom.

R⁴ is preferably a hydrogen atom or an alkyl or alkenyl group having from 1 to 4 carbon atoms. Most preferably R⁴ is a hydrogen atom.

R⁵ is preferably a hydrogen atom or an alkyl or alkenyl group having from 1 to 4 carbon atoms. Most preferably R⁵ is a hydrogen atom.

In some embodiments three of the entities R², R³, R⁴ and R⁵ are hydrogen and only one is an alkyl or alkenyl group having from 1 to 8 carbon atoms. In such embodiments preferably R³, R⁴ and R⁵ are hydrogen and R² is the alkyl or alkenyl group.

However in especially preferred embodiments R², R³, R⁴ and R⁵ are all hydrogen

In some especially favoured embodiments component (a) comprises one or both of sodium lauroyl isethionate (SLI), sodium oleoyl isethionate and sodium cocoyl isethionate (SCI), which is closely related to but not identical to SLI.

Most preferably the component (a) of the composition of the present invention comprises or consists of sodium cocoyl isethionate and/or sodium lauroyl isethionate.

An acyl isethionate may serve as the only constituent of component (a). However in some acceptable embodiments component (a) may comprise an acyl isethionate in admixture with one or more further non-soap anionic surfactants, for example any of those defined above. In such admixture embodiments acyl isethionate(s) suitably constitute(s) at least 50% wt of, and preferably at least 65% wt, of component (a).

Suitably the non-soap anionic surfactant, component (a), constitutes at least 15% wt of the solid cleansing composition.

Suitably in some embodiments the non-soap anionic surfactant constitutes at least 16% wt of the solid cleansing composition.

Suitably in some embodiments the non-soap anionic surfactant constitutes at least 18% wt of the solid cleansing composition.

Suitably in some embodiments the non-soap anionic surfactant constitutes at least 20% wt of the solid cleansing composition.

Suitably in some embodiments the non-soap anionic surfactant constitutes up to 40% wt of the solid cleansing composition.

Suitably the non-soap anionic surfactant constitutes up to 36% wt of the solid cleansing composition.

Suitably the non-soap anionic surfactant constitutes up to 32% wt of the solid cleansing composition.

Suitably in some embodiments the non-soap anionic surfactant constitutes up to 28% wt of the solid cleansing composition.

Suitably in some embodiments the non-soap anionic surfactant constitutes from 16 to 32% wt of the solid cleansing composition, suitably from 18 to 28% wt, suitably from 18 to 24% wt of the solid cleansing composition.

Suitably the non-soap anionic surfactant constitutes at least 14% wt of the emulsion.

Suitably in some embodiments the non-soap anionic surfactant constitutes at least 15% wt of the emulsion.

Suitably in some embodiments the non-soap anionic surfactant constitutes at least 17% wt of the emulsion.

Suitably in some embodiments the non-soap anionic surfactant constitutes at least 18% wt of the emulsion.

Suitably in some embodiments the non-soap anionic surfactant constitutes up to 38% wt of the emulsion.

Suitably the non-soap anionic surfactant constitutes up to 36% wt of the emulsion.

Suitably the non-soap anionic surfactant constitutes up to 32% wt of the emulsion.

Suitably the non-soap anionic surfactant constitutes up to 28% wt of the emulsion.

Suitably in some embodiments the non-soap anionic surfactant constitutes up to 25% wt of the emulsion.

Suitably in some embodiments the non-soap anionic surfactant constitutes from 14 to 30% wt of the emulsion, suitably from 16 to 26% wt, suitably from 16 to 25% wt of the emulsion.

Suitably component (b) may have a melting point of at least 50° C., preferably of at least 55° C., more preferably of at least 60° C. In some preferred embodiments component (b) may have a melting point of at least 65° C.

Suitably the carrier, component (b), is selected from a triglyceride, a fatty acid, a fatty alcohol or a wax (including a mixture of any of these components).

Suitably the carrier, component (b), may be beeswax, or may be a triglyceride, fatty acid, fatty alcohol or wax of vegetal origin.

Suitably component (b) may have a melting point of at least 50° C., preferably of at least 55° C., more preferably of at least 60° C. In some preferred embodiments component (b) may have a melting point of at least 65° C.

Preferably component (b) is a triglyceride whose fatty acid chains are residues of a C₈ fatty acid, or of higher fatty acids. Preferably they are residues of C₈-C₃₂ fatty acids, preferably of C₁₀-C₂₄ fatty acids, preferably of C₁₂-C₂₀ fatty acids and most preferably of C₁₂₋₁₆ fatty acids.

Preferably component (b) is a saturated triglyceride. Preferably substantially all fatty acid chains in the triglyceride are saturated. Preferably component (b) is a hydrogenated triglyceride.

Suitably component (b) comprises at least of one of hydrogenated rapeseed oil, hydrogenated soybean oil, hydrogenated sunflower oil, hydrogenated palm oil, hydrogenated castor oil, hydrogenated safflower oil and hydrogenated peanut oil. Such oils may be employed singly or in admixture of two, three, four or all five of these hydrogenated oils. Suitably hydrogenated rapeseed oil, hydrogenated soybean oil, hydrogenated sunflower oil, hydrogenated palm oil, hydrogenated castor oil, hydrogenated safflower oil and hydrogenated peanut oil (summated if more than one is used) make up at least 50% wt, preferably at least 60% wt, preferably at least 70% wt, preferably at least 80% wt, preferably at least 90% wt, and preferably 100% wt, of the carrier, component (b).

Suitable carriers also include fatty acids, suitably C₈ fatty acid, or higher fatty acids; for example C₈-C₃₂ fatty acids, preferably C₁₀-C₂₄ fatty acids, preferably C₁₂-C₂₀ fatty acids and most preferably C₁₂-C₁₆ fatty acids.

Suitable carriers also include fatty alcohols, suitably C₁₆ fatty alcohol, or higher fatty alcohol; for example C₈-C₃₂ fatty alcohols, preferably C₁₀-C₂₄ fatty alcohols, preferably C₁₂-C₂₀ fatty alcohols and most preferably C₁₆-C₁₈ fatty alcohols.

A fatty acid or fatty alcohol residue Cₓₓ herein denotes a fatty acid or fatty alcohol residue in which Cₓₓ represents the mean of the residues or in which residues Cₓₓ constitute more than half the weight of total fatty acid or fatty alcohol residues. If either of these definitions is satisfied the definitions given herein apply.

Suitable waxes include waxes of vegetal origin, for example carnauba wax, and beeswax.

Suitably the carrier, component (b), constitutes at least 8% wt of the solid cleansing composition.

Suitably the carrier constitutes at least 10% wt of the solid cleansing composition.

Suitably in some embodiments the carrier constitutes at least 12% wt of the solid cleansing composition.

Suitably in some embodiments the carrier constitutes at least 14% wt of the solid cleansing composition.

Suitably in some embodiments the carrier constitutes at least 16% wt of the solid cleansing composition.

Suitably the carrier constitutes up to 36% wt of the solid cleansing composition.

Suitably the carrier constitutes up to 32% wt of the solid cleansing composition.

Suitably the carrier constitutes up to 28% wt of the solid cleansing composition.

Suitably the carrier constitutes up to 24% wt of the solid cleansing composition.

Suitably the carrier constitutes up to 22% wt of the solid cleansing composition.

Suitably the carrier, component (b), constitutes from 10 to 30% wt of the solid cleansing composition, suitably from 14 to 24% wt, suitably from 16 to 22% wt of the solid cleansing composition.

Suitably in some embodiments the carrier constitutes at least 10% wt of the emulsion.

Suitably in some embodiments the carrier constitutes at least 12% wt of the emulsion.

Suitably in some embodiments the carrier constitutes at least 14% wt of the emulsion.

Suitably in some embodiments the carrier constitutes at least 16% wt of the emulsion.

Suitably the carrier constitutes up to 32% wt of the emulsion.

Suitably the carrier constitutes up to 28% wt of the emulsion.

Suitably the carrier constitutes up to 26% wt of the emulsion.

Suitably the carrier constitutes up to 24% wt of the emulsion.

Suitably the carrier constitutes up to 21% wt of the emulsion.

Suitably the carrier, component (b), constitutes from 10 to 30% wt of the emulsion, suitably from 14 to 24% wt, suitably from 16 to 22% wt of the emulsion.

Suitably the ratio by weight of the non-soap anionic surfactant (a) to the carrier (b), (a):(b), is in the range 80 parts (a):100 parts (b) to 100 parts (a):60 parts (b).

Suitably the ratio by weight of the non-soap anionic surfactant (a) to the carrier (b), (a):(b), is in the range 90 parts (a):100 parts (b) to 100 parts (a):70 parts (b).

Suitably the ratio by weight of the non-soap anionic surfactant (a) to the carrier (b), (a):(b), is in the range 100 parts (a):100 parts (b) to 100 parts (a):80 parts (b).

Preferably the mass of the non-soap anionic surfactant (a) equals or exceeds the mass of the carrier (b). These definitions apply to the solid cleansing composition and to the emulsion.

Suitably the emulsifier, component (c), has an HLB value of at least 5, suitably at least 6, preferably at least 7, and most preferably at least 8.

Suitably the emulsifier has an HLB value of up to 12, preferably up to 11, and most preferably up to 10.

Thus a suitable range for the emulsifier is from 5 to 12 or from 6 to 12, and a preferred range is from 8 to 10.

The desired HLB value may be that of a single emulsifier or may be achieved by selection of two or more emulsifiers which in combination—calculated as a weighted average taking into account their individual HLB values and their amounts in the blend—achieve the desired HLB value, within the preferred range 6-12. Thus, a weighted average for emulsifiers A¹, A², A³ . . . can be calculated using the equation:

$$\frac{\begin{matrix}( mass of {A}^{1} \times HLB of {A}^{1} ) + ( mass of {A}^{2} \times HLB of {A}^{2} ) + \\ ( mass of {A}^{3} \times HLB of {A}^{3} ) + …\end{matrix}}{mass of {A}^{1} + mass of {A}^{2} + mass of {A}^{3} + …}$$

Suitable emulsifiers (c) include sorbitan fatty acid esters and ethoxylated sorbitan fatty acid esters. Suitable sorbitan fatty acid esters, non-ethoxylated, include sorbitan monolaurate, sorbitan monooleate, sorbitan monopalmitate, sorbitan monostearate and sorbitan tristearate. Suitable ethoxylated sorbitan fatty acid esters include polyoxyethylene sorbitan monolaurate, polyoxyethylenesorbitan monooleate, polyoxyethylene sorbitan monopalmitate, polyoxyethylene sorbitan monostearate and polyoxyethylene sorbitan tristearate.

Suitable emulsifiers (c) include glycerol esters of fatty acids; that is, esters of fatty acids and glycerol or polyglycerol. A very wide range of suitable glycerol esters may be made by reaction of glycerol or polyglycerol and C₆-C₃₆ fatty acids, for example C₁₂-C₂₄ fatty acids. The fatty acids may be supplied to the esterification reaction as fatty acids or as precursor ester compounds, for example triglycerides. Examples suitable for use in this invention include glycerol monostearate, glycerol monooleate, glycerol palmitate and glycerol monolaurate.

It has been found useful to employ a blend of a glycerol/polyglycerol ester of a fatty acid and a sorbitan fatty acid ester or a polyethoxylated sorbitan fatty acid ester, in order to achieve (as a weighted average) an HLB value as defined above for the emulsifier (c).

We have found that one particular sub-class of glycerol/polyglycerol ester, described below, is especially beneficial in the present invention.

In such embodiments of the invention the emulsifier, component (c), may be the esterification reaction product of A) a triglyceride, or a fatty acid, or a methyl ester of a fatty acid (or any combination thereof) and B) a polyhydric alcohol which comprises B1) a polyglycerol having at least 3 glycerol units, and B2) glycerol and/or diglycerol.

Preferably the triglyceride A) is an oil (being a liquid at ambient temperature of 18° C.) of plant origin. Preferably it is a naturally-occurring oil, or fat, whose structure has not been chemically modified prior to the esterification reaction.

Batches of triglycerides may vary in their average molecular weight and in their distribution of components but the values stated in this specification define typical compositions suitable for use in the present invention.

The triglyceride, or the parent triglyceride of the fatty acid or methyl ester of a fatty acid, may be from a single triglyceride source, for example a single named oil or fat, or from a plurality of triglyceride sources, blended.

The fatty acid moieties of the triglyceride, or of the fatty acid or of the methyl ester of a fatty acid, each have an R—CO— moiety, where R represents a hydrocarbyl moiety. Preferably there are at least 6 carbon atoms in the R—CO— moiety, preferably at least 8 carbon atoms; preferably at least 10 carbon atoms; most preferably at least 12 carbon atoms. Preferably there are up to 36 carbon atoms in the R—CO— moiety, preferably up to 28 carbon atoms, preferably up to 24 carbon atoms, more preferably up to 20 carbon atoms.

Suitable fats or oils for use in the esterification reaction may be selected from one or more of the following: almond oil, babassu oil, borage oil, canola oil, cocoa butter, coconut oil, corn oil (maize oil), cottonseed oil, flaxseed oil, grape seed oil, hazelnut oil, illipe, oat oil, olive oil, palm oil, palm olein, palm kernel oil, peanut oil, rapeseed oil, safflower oil, sesame oil, shea nut, soybean oil, tucum oil, sunflower oil, walnut oil, apricot oil, sweet almond oil, avocado oil, baobab oil, blueberry seed oil, calendula oil, camellia oil, cherry kernel oil, cranberry seed oil, hemp oil, jojoba oil, kukur nut oil, macadamia nut oil, manketti oil, melon seed oil, moringe oil, peach kernel oil, pistachio oil, raspberry seed oil, rice bran oil, rosehip oil, soya oil, wheatgerm oil, yangu oil; and their hydrogenated derivatives. A blend of oils or fats may be employed.

Fatty acids or methyl esters of fatty acids, which may be used in the esterification reaction, may include fatty acids or methyl esters derived from any of the fats or oils described above.

Fatty acids which may be used in the esterification reaction, or which may be regarded as ‘delivered’ to the esterification reaction by a fat or oil, may be selected from one or more of the following: caproic acid, caprylic acid, capric acid, lauric acid, myristic acid, palmitic acid, palmitoleic acid, stearic acid, oleic acid, ricinoleic acid, vaccenic acid, linoleic acid, alpha-linolenic acid (ALA), gamma-linolenic acid (GLA), arachidic acid, gadoleic acid, arachidonic acid (AA), EPA (5,8,11,14,17-eicosapentaenoic acid), behenic acid, erucic acid, DHA (4,7,10,13,16,19-docosahexaenoic acid), and lignoceric acid; and methyl esters of such acids. A blend of fatty acids and/or fatty acid methyl esters may be employed.

Especially preferred acids include caproic acid, caprylic acid, capric acid, lauric acid, myristic acid, palmitic acid, stearic acid, linoleic acid, alpha-linolenic acid (ALA)—and gamma-linoleic acid (GLA). Especially preferred methyl esters may be methyl esters of such fatty acids. Oils or fats which yield such fatty acids are preferred oils or fats. Especially preferred oils are flax seed (flax) oil (which can yield alpha-linolenic acid, ALA), borage seed (borage) oil (which can yield gamma-linolenic acid, GLA) and palm kernel oil (which can yield palmitic acid).

The fatty acid moieties, whether as free acids or methyl esters thereof or as components of a triglyceride, may be saturated or unsaturated. If unsaturated they may suitably have from 1 to 6 double bonds, preferably 1 to 3 double bonds. The fatty acids or methyl esters or parent oils or fats may have been hydrogenated.

In one embodiment the fatty acid moieties, whether free acids or methyl esters thereof or as a component of a triglyceride, are saturated fatty acid moieties having an average molecular weight of less than 282 g/mol, preferably less than 260 g/mol, preferably less than 240 g/mol.

In one embodiment the fatty acid moieties, whether free acids or methyl esters thereof or as a component of a triglyceride, are unsaturated fatty acid moieties having an average molecular weight of less than 320 g/mol, preferably less than 300 g/mol.

Batches of polyglycerols may vary in their average molecular weight and in their distribution of components but the values stated in this specification define typical situations or typical average values.

Preferably the polyglycerol component B1) comprises polyglycerol-4.

Polyglycerol-4 is a commercially available product sold under that name. Polyglycerol-4 is a distribution of a polyglycerol compounds in which the average molecular weight of the polyglycerol is centred on polyglycerol-4.

Preferably the polyglycerol component B1) constitutes at least 50% wt of polyglycerol compounds composed of 3 to 6 glycerol units, preferably at least 60% wt, preferably at least 70% wt, most preferably at least 85% wt.

Preferably the polyglycerol component B1) constitutes at least 20% wt of the compound tetraglycerol, preferably at least 25% wt.

Suitably the emulsifier, component (c), is an esterification reaction product of a) a triglyceride and b) a polyhydric alcohol which comprises b1) a polyglycerol having at least 3 glycerol units, and b2) glycerol, wherein the polyglycerol component b1) comprises at least 25 wt % of the compound tetraglycerol.

Triglycerol may also be a significant component, even when the average molecular weight of the polyglycerol is centred on polyglycerol-4. Preferably the polyglycerol component B1) constitutes at least 20% wt of the compound triglycerol, preferably at least 25% wt.

Suitably the emulsifier, component (c), is an esterification reaction product of a) a triglyceride and b) a polyhydric alcohol which comprises b1) a polyglycerol having at least 3 glycerol units, and b2) glycerol, wherein the polyglycerol component b1) comprises at least 25 wt % of the compound tetraglycerol and at least 25 wt % of the compound triglycerol.

Preferably the compound tetraglycerol is present in the polyglycerol component B1) in a higher amount by weight than the compound pentaglycerol.

Preferably the compound tetraglycerol is the single compound present in the largest proportion by weight in the polyglycerol component B1).

In commercial sources of polyglycerol-4, which may be used in the present invention as a preferred component B1), the proportions by weight of compounds is typically as follows:
triglycerol, 20-50% wt
tetraglycerol, 20-50% wt
higher polyglycerols, 15-40% wt in total
other compounds, not more than 20% wt, typically less than 12% wt.

Higher polyglycerols herein means polyglycerol compounds formed of 5 or more glycerol units.

The other compounds may include water and diglycerol. Small amounts of glycerol may sometimes be present. However glycerol need not be present in the emulsifier (c).

When glycerol and/or diglycerol is/are present in the polyglycerol source they may supply some or all of component B2). However the amount of glycerol and diglycerol in a polyglycerol source is generally low. Therefore, in such embodiments, it is generally required to supply glycerol and/or diglycerol to reach the desired amount. This may be done by simple addition of glycerol and/or diglycerol. When a triglyceride is used it may occur by the liberation of glycerol in the esterification reaction. Suitably when a triglyceride is used, no glycerol is added and substantially all the glycerol used in the reaction is glycerol liberated from the triglyceride during the reaction.

Preferably the average molecular weight of the polyglycerols in component B1), having at least 3 glycerol units, is in the range 220-500 g/mol, preferably 230-450 g/mol, preferably 240-420 g/mol, preferably 250-400 g/mol, preferably, preferably 260-370 g/mol, 280-350 g/mol, most preferably 300-330 g/mol.

Preferably glycerol constitutes at least 60% wt of the component B2), preferably at least 70% wt, preferably at least 80% wt.

Preferably the ratio by weight of the component B2) to component B1) is one part B2) to at least 4 parts B1), preferably one part B2) to at least 6 parts B1), and most preferably at least one part B2) to at least 8 parts B1).

Preferably the ratio by weight of the component B2) to component B1) is one part B2) to up to 24 parts B1), preferably one part B2) to up to 20 parts B1), and most preferably one part B2) to up to 16 parts B1).

Preferably the components B1) and B2) make up at least 50% wt of the functional fluid, preferably at least 60% wt, preferably at least 70% wt, preferably at least 80% wt, preferably at least 90% wt.

When a fatty acid or fatty acid methyl ester is used for the esterification reaction to make component (c), component B2) is added to the reaction mixture, suitably in such an amount as to satisfy at least one of the B1):B2) definitions given above.

When a triglyceride is used, preferably the molar ratio of the polyglycerol to the triglyceride in the reaction mixture, based on average molecular weights, is 0.5-5 to 1, preferably 1-4 to 1, preferably 1.5-3 to 1, most preferably 2-2.5 to 1. Average molecular weight may be determined by normal measures. In the case of the triglyceride this may be from GC results. In the case of polyglycerol the raw material specifications may be used.

When a fatty acid or derivative is used, the molar ratio of the polyglycerol to the fatty acid in the reaction mixture, based on average molecular weights, is 0.2-3 to 1, preferably 0.3-2 to 1, preferably 0.5-1.5 to 1, most preferably 0.7-1.3 to 1.

“Esterification” herein includes transesterification or interesterification.

Standard esterification conditions may be used for the manufacture of the glycerol ester emulsifier. Suitably reactions which employ triglycerides are carried out under alkaline conditions (e.g. by addition of sodium hydroxide or potassium hydroxide) and at elevated temperature, for example at least 150° C., preferably 200-250° C. Suitably reactions which employ fatty acids added to the reaction mixture are carried out under acidic conditions (e.g. by addition of sulphonic acid or orthophosphoric acid) and at elevated temperature, for example at least 150° C., preferably 200-250° C.

Preferably the process is a one-pot process, even when it employs a triglyceride.

Preferably the emulsifier is the product of the esterification reaction, without work up (other than neutralisation of any catalyst present). Preferably it is a liquid at ambient temperature of 18° C.

Preferably the emulsifier comprises a plurality of esters, formed from the range of triglycerides present in oil or fat (or from the range of fatty or methyl esters obtained from such triglycerides), and the range of polyglycerol compounds present in an as-supplied “polyglycerol” source. The emulsifier may comprise many esters, for example over 20 ester compounds, or over 50 esters, or over 100 esters.

Suitably the emulsifier, component (c), is an esterification reaction product, without work-up other than neutralisation of any catalyst present, of a) a triglyceride and b) a polyhydric alcohol which comprises b1) a polyglycerol having at least 3 glycerol units, and b2) glycerol and/or diglycerol,

wherein the polyglycerol component b1) comprises:
triglycerol, 25-50% wt
tetraglycerol, 25-50% wt
higher polyglycerols, 15-40% wt
other compounds, not more than 20% wt;
   wherein the molar ratio of component b1) to component a) in the esterification reaction mixture, based on average molecular weights, is 1-4 to 1; and
   wherein the esterification reaction is carried out under alkaline conditions at a temperature of at least 150° C.

By use of the methods described herein, emulsifiers having an HLB value in the ranges stated above, or able to provide a weighted average HLB value when mixed with other emulsifiers, may be made and are found to be suitably for use in the present invention.

Suitably the emulsion is an oil-in-water emulsion.

Suitably the emulsifier, component (c), constitutes at least 3% wt of the solid cleansing composition.

Suitably the emulsifier constitutes at least 4% wt of the solid cleansing composition.

Suitably the emulsifier constitutes at least 5% wt of the solid cleansing composition.

Suitably the emulsifier constitutes at least 6% wt of the solid cleansing composition.

Suitably the emulsifier constitutes at least 7% wt of the solid cleansing composition.

Suitably the emulsifier constitutes at least 7.5% wt of the solid cleansing composition.

The emulsifier may in some embodiments constitute up to 14% wt of the solid cleansing composition.

The emulsifier may in some embodiments constitute up to 12% wt of the solid cleansing composition.

The emulsifier may in some embodiments constitute up to 10% wt of the solid cleansing composition.

Suitably the emulsifier constitutes from 4 to 14% wt of the solid cleansing composition, suitably from 6 to 12% wt, suitably from 7 to 11% wt of the solid cleansing composition.

Suitably the emulsifier constitutes at least 3% wt of the emulsion.

Suitably the emulsifier constitutes at least 4% wt of the solid cleansing composition.

Suitably the emulsifier, component (c), constitutes at least 5% wt of the emulsion.

Suitably the emulsifier, component (c), constitutes at least 6% wt of the emulsion.

Suitably the emulsifier constitutes at least 7% wt of the emulsion.

In some embodiments the emulsifier constitutes up to 14% wt of the emulsion.

In some embodiments the emulsifier constitutes up to 12% wt of the emulsion.

In some embodiments the emulsifier constitutes up to 10% wt of the emulsion.

In some embodiments the emulsifier constitutes up to 9% wt of the emulsion.

Suitably the emulsifier constitutes from 4 to 14% wt of the emulsion, suitably from 6 to 12% wt, suitably from 7 to 11% wt of the emulsion.

Preferably the humectant (d) is a humectant polyhydric alcohol or a humectant ester or humectant ether thereof.

Preferably the humectant (d) is selected from compounds having from 2 to 6 hydroxyl groups and their di-, tri-, and oligomeric forms.

Preferably the humectant (d) is selected from one or more of: glycerol; diglycerol; triglycerol; polyoxy ethylene glycerol; alpha methyl glycerol; ethylene glycol, diethylene glycol, triethylene glycol and higher polyethylene glycols; monopropylene glycol, dipropylene glycol, tripropylene glycol and higher polypropylene glycols; butylene glycol, dibutylene glycol, tributylene glycol and higher polybutylene glycols; hexane diol; 1,2,6-hexanetriol; hexylene glycol; capryl glycol; phytantriol; sugar alcohols, for example mannitol, sorbitol xylitol and erythritol; glucose; propylene glycol glucoside; urea; triethanolamine lactate.

A preferred humectant is glycerol. Benefit may be obtained from using glycerol with monopropylene glycol (MPG), preferably in an amount of 80 parts or more glycerol:20 parts or less MPG; preferably 90 parts or more glycerol:10 parts or less MPG (by weight).

Suitably the humectant, component (d), constitutes at least 12% wt of the solid cleansing composition.

Suitably in some embodiments the humectant constitutes at least 14% wt of the solid cleansing composition.

Suitably in some embodiments the humectant constitutes at least 16% wt of the solid cleansing composition.

Suitably in some embodiments the humectant constitutes at least 18% wt of the solid cleansing composition.

Suitably in some embodiments the humectant constitutes at least 20% wt of the solid cleansing composition.

Suitably in some embodiments the humectant constitutes at least 22% wt of the solid cleansing composition.

Suitably in some embodiments the humectant constitutes at least 24% wt of the solid cleansing composition.

Suitably the humectant constitutes up to 40% wt of the solid cleansing composition.

Suitably the humectant constitutes up to 36% wt of the solid cleansing composition.

Suitably the humectant constitutes up to 32% wt of the solid cleansing composition.

Suitably the humectant constitutes up to 30% wt of the solid cleansing composition.

Suitably the humectant constitutes from 16 to 40% wt of the solid cleansing composition, suitably from 18 to 36% wt, suitably from 22 to 32% wt, suitably from 22 to 28% wt of the solid cleansing composition.

Suitably in some embodiments the humectant constitutes at least 12% wt of the emulsion.

Suitably in some embodiments the humectant constitutes at least 14% wt of the emulsion.

Suitably in some embodiments the humectant constitutes at least 16% wt of the emulsion.

Suitably in some embodiments the humectant constitutes at least 18% wt of the emulsion.

Suitably in some embodiments the humectant constitutes at least 20% wt of the emulsion.

Suitably in some embodiments the humectant constitutes at least 22% wt of the emulsion.

Suitably the humectant constitutes up to 40% wt of the emulsion.

Suitably the humectant constitutes up to 36% wt of the emulsion.

Suitably the humectant constitutes up to 32% wt of the emulsion.

Suitably the humectant constitutes up to 30% wt of the emulsion.

Suitably the humectant constitutes up to 28% wt of the emulsion.

Suitably the humectant constitutes up to 26% wt of the emulsion.

Suitably the humectant constitutes from 16 to 40% wt of the emulsion, suitably from 18 to 36% wt, suitably from 22 to 32% wt, suitably from 22 to 28% wt of the emulsion.

Suitably the ratio by weight of the humectant (d) to the emulsifier (c), in the solid cleansing composition and the emulsion is in the range 600 parts (d):100 parts (c).

Suitably the ratio by weight of the humectant (d) to the emulsifier (c) is in the range 500 parts (d):100 parts (c).

Suitably the ratio by weight of the humectant (d) to the emulsifier (c) is in the range 400 parts (d):100 parts (c).

It has been explained that a preferred emulsifier, component (c) may contain a small amount of glycerol or other polyol. Glycerol is defined herein as a preferred humectant, component (d). The definitions given in this specification for component (d) refer only to the glycerol (or other polyol) added as the humectant, and do not include the glycerol (or other polyol) which may be present as part of component (c).

Suitably water, component (e), constitutes at least 10% wt of the solid cleansing composition.

Suitably water constitutes at least 12% wt of the solid cleansing composition.

Suitably water constitutes at least 14% wt of the solid cleansing composition.

Suitably water constitutes at least 16% wt of the solid cleansing composition.

Suitably water constitutes at least 18% wt of the solid cleansing composition.

Suitably water constitutes at least 20% wt of the solid cleansing composition.

Suitably water constitutes up to 36% wt of the solid cleansing composition.

Suitably water constitutes up to 34% wt of the solid cleansing composition.

Suitably water constitutes up to 32% wt of the solid cleansing composition.

Suitably water constitutes up to 30% wt of the solid cleansing composition.

Suitably water constitutes up to 28% wt of the solid cleansing composition.

Suitably water constitutes up to 26% wt of the solid cleansing composition.

Suitably water constitutes up to 24% wt of the solid cleansing composition.

Suitably water constitutes up to 22% wt of the solid cleansing composition.

Suitably water constitutes from 16 to 36% wt of the solid cleansing composition, suitably from 20 to 30% wt, suitably from 22 to 28% wt of the solid cleansing composition.

Suitably water, component (e), constitutes at least 12% wt of the emulsion.

Suitably water constitutes at least 14% wt of the emulsion.

Suitably water constitutes at least 16% wt of the emulsion.

Suitably water constitutes at least 17% wt of the emulsion.

Suitably water constitutes at least 18% wt of the emulsion.

Suitably water constitutes at least 20% wt of the emulsion.

Suitably water constitutes at least 22% wt of the emulsion.

Suitably water constitutes at least 23% wt of the emulsion.

Suitably water constitutes up to 40% wt of the emulsion.

Suitably water constitutes up to 38% wt of the emulsion.

Suitably water constitutes up to 36% wt of the emulsion.

Suitably water constitutes up to 34% wt of the emulsion.

Suitably water constitutes up to 32% wt of the emulsion.

Suitably water constitutes up to 30% wt of the emulsion.

Suitably water constitutes up to 28% wt of the emulsion.

Suitably water constitutes up to 26% wt of the emulsion.

Suitably water constitutes from 16 to 36% wt of the emulsion, suitably from 20 to 30% wt, suitably from 22 to 28% wt of the emulsion.

It is possible that some components (a) to (d) may provide small amounts of water. For example component (a) may be a product supplied with a small amount of water. Water is defined herein as component (e). The definitions given in this specification for component (e) refer to total water content, whether added as water itself or as part of another component.

Suitably the ratio by weight of the humectant (d) to water (e) in the emulsion, (d):(e), is in the range 60 parts (d):100 parts (e) to 100 parts (d):60 parts (e).

Suitably the ratio by weight of the humectant (d) to water (e) in the emulsion, (d):(e), is in the range 70 parts (d):100 parts (e) to 100 parts (d):70 parts (e).

Suitably the ratio by weight of the humectant (d) to water (e) in the emulsion, (d):(e), is in the range 80 parts (d):100 parts (e) to 100 parts (d):80 parts (e).

Suitably the ratio by weight of the humectant (d) to water (e) in the emulsion, (d):(e), is in the range 90 parts (d):100 parts (e) to 100 parts (d):90 parts (e).

Preferably the mass of humectant (d) equals or exceeds the mass of water (e), in the emulsion.

Suitably the ratio by weight of the humectant (d) to water (e) in the cleansing composition, (d):(e), is in the range 50 parts (d):100 parts (e) to 100 parts (d):80 parts (e).

Suitably the ratio by weight of the humectant (d) to water (e) in the cleansing composition, (d):(e), is in the range 60 parts (d):100 parts (e) to 100 parts (d):80 parts (e).

Suitably the ratio by weight of the humectant (d) to water (e) in the cleansing composition, (d):(e), is in the range 70 parts (d):100 parts (e) to 100 parts (d):90 parts (e).

Suitably the ratio by weight of the humectant (d) to water (e) in the cleansing composition, (d):(e), is in the range 80 parts (d):100 parts (e) to 100 parts (d):100 parts (e).

Preferably the mass of humectant (d) exceeds the mass of water (e), in the solid cleansing composition.

Optional further components may include (without limitation) chelants, fatty alcohols and glycols and polyols not having hygroscopic properties, amphoteric surfactants and preservatives. Further components may offer manufacturing benefits, for example foam control during manufacture, and/or end product user benefits, for example foam control during washing, or emolliency. Such further components may preferably be present in total in an amount of up to 30% wt of the solid cleansing composition.

Suitable chelants include DTPA (diethylenetriaminepentaacetic acid), HEDP (hydroxyethylidene diphosphonic acid), NTA (nitrilotriacetic acid), EDTA (ethylenediaminetetraacetic acid), EDDS (ethylenediamine-N,N′-disuccinic acid), GLDA (N,N-dicarboxymethyl glutamic acid tetrasodium salt) and PDTA (propylenedinitrilotetraacetic acid); and chelants which are analogues of any of the foregoing. Chelants, when present, may suitably be present in total in an amount of up to 0.5% wt of the solid cleansing composition, preferably up to 0.2% wt.

Amphoteric surfactants include betaines, for example cocamidopropyl betaine (CAPB). Amphoteric surfactants, when present, may suitably be present in total in an amount of up to 10% wt of the solid cleansing composition, preferably up to 6% wt.

Suitable preservatives include sorbic acid, sorbates (for example potassium sorbate), benzoic acid, benzoates (for example sodium benzoate); organohalogen compounds (for example Triclosan), aldehydes, glycol ethers (for example phenoxyethanol, 2-butoxyethanol, 2-(2-butoxyethoxy)-ethanol2-(2-ethoxy)-ethanol; and parabens (for example methylparaben, ethylparaben, propylparaben, butylparaben and isobutylparaben).

The pH of the solid cleansing composition of the present invention (measured at 5 wt % dilution into water) is preferably less than 8, more preferably less than 7, more preferably less than 6.5. Suitably the pH (measured at 10% wt in water) of the solid cleansing composition is greater than 4, and preferably greater than 5, and preferably greater than 5.5.

In some preferred embodiments the solid composition comprises:
(a) at least 16% wt and up to 36% wt, of a non-soap anionic surfactant having a melting point of at least 45° C.;
(b) at least 14% wt and up to 28% wt, of a carrier having a melting point of at least 60° C.;
(c) at least 6% wt and up to 12% wt, of an emulsifier;
(d) at least 14% wt and up to 32% wt, of an humectant;
(e) at least 16% wt and up to 32% wt, of water;
   and preferably does not contain soap or starch;
   wherein components (a) to (e) make up at least 70% of the weight of the solid cleansing composition. Any further components, when present, are preferably selected from the further components described above.

In further preferred embodiments the solid composition comprises:
(a) at least 20% wt and up to 28% wt, of an isethionate non-soap anionic surfactant having a melting point of at least 45° C.;
(b) at least 18% wt and up to 22% wt, of a hydrogenated vegetable oil carrier having a melting point of at least 60° C.;
(c) at least 6% wt and up to 10% wt, of a polyglyceryl emulsifier;
(d) at least 22% wt and up to 30% wt, of glycerol;
(e) at least 18% wt and up to 28% wt, of water;
   and preferably does not contain soap or starch;
   wherein components (a) to (e) make up 80-100% of the weight of the solid cleansing composition. Any further components, when present, may suitably be selected from the further components described above.

Preferably the solid cleansing composition of the first aspect is a solid throughout the range from 0 to 40° C. Preferably it is homogenous; no particulates can be seen when a cut surface is examined under an optically microscope at 100× magnification. The homogenous nature is the result of a molten process for preparation of the solid cleansing composition.

A solid cleansing composition in block form, having ingredients as defined above in amounts as defined above, is suitably manufactured by preparing an emulsion of the ingredients at an elevated temperature, cooling the emulsion, and allowing it to solidify into a block particulate mass, for later re-melting and casting into individual tablets or bars for personal use.

It will be understood by the skilled reader that components of the compositions of the invention—notably components (a) and (b)—may be derived from natural sources and may contain a distribution of molecules about a mean carbon value. Such components are expected to melt over a temperature range, rather than have a sharp melting point. The melting point definitions defined in this specification refer to the temperatures at which a material is completely melted. The method described in the paper “Determination of melting point of vegetable oils and fats by differential scanning calorimetry (DSC) technique” by Renata Tieko Nassu and Lireny Aparecida Guaraldo Gonsalves, in the Grasas y Aceites Vol. 50. Fase. 1 (1999), 16-22 may be consulted. DSC as described in this paper can be used to determine when a material is completely in the liquid state.

In some embodiments, the solid composition may comprise, in component (b), one or more waxes, for example to increase the hardness of the solid cleansing composition.

Suitable waxes include waxes of vegetal origin, for example carnauba wax, beeswax or sunflower wax. The wax may be cetearyl alcohol.

Therefore the present invention may provide a solid cleansing composition comprising:
(a) at least 12% wt of a non-soap anionic surfactant having a melting point of at least 45° C.;
(b) at least 8% wt of a carrier having a melting point of at least 45° C.;
(c) at least 3% wt of an emulsifier;
(d) at least 8% wt of a humectant; and
(e) at least 8% wt water;
(f) a second emulsifier
   wherein the composition is a solid throughout the range from 0° C. to 40° C.;
   wherein component (b) comprises one or more waxes.

Suitably component (b) comprises an oil as defined above and the one or more waxes. Suitably component (b) comprises a triglyceride or a hydrogenated triglyceride as defined above and the one or more waxes. For example, component (b) may comprise sunflower wax or cetearyl alcohol. Suitably component (b) comprises sunflower wax and cetearyl alcohol.

In such embodiments, the carrier (b) in total constitutes at least 10% wt and up to 32% wt of the solid cleansing composition and the one or more waxes suitably provide from 1 to 10% wt of the solid cleansing composition, suitably from 2 to 8% wt, suitably from 3 to 7% wt of the solid cleansing composition. Suitably the one or more waxes in the carrier (b) provide from 4 to 6% wt of the solid cleansing composition. Suitably the solid cleansing composition comprises said amounts of the one or more waxes and from 10 to 30% wt of an oil, suitably from 12 to 25% wt, suitably from 14 to 20% wt of an oil, suitably a triglyceride or a hydrogenated triglyceride as defined above, for example hydrogenated sunflower oil.

Suitably the component (b) provides from 16 to 28% wt of the solid cleansing composition; wherein the one or more waxes provides from 2 to 8% wt and the oil, suitably a triglyceride or a hydrogenated triglyceride, provides from 14 to 20% wt of the solid cleansing composition.

In such embodiments, component (c) the emulsifier suitably comprises a first emulsifier and a second emulsifier. Suitably the first emulsifier is as defined above, suitably having an HLB value of from 8 to 10. Suitably the second emulsifier has an HLB value of from 3 to 7, suitably from 3 to 6 or from 4 to 6. Therefore component (c) suitably comprises a first emulsifier having an HLB value of from 8 to 10 and a second emulsifier having an HLB value of from 3 to 6.

Suitably component (c) consists essentially or consists of the first and second emulsifiers.

Suitably the component (c) comprising the first emulsifier and the second emulsifier has a combined HLB value of from 5 to 8, suitably from 5 to 7, for example from 5 to 6 (when the first and second emulsifiers are considered together).

The inventors have found that this combination of first and second emulsifiers having the different HLB ranges discussed above can be used with the one or more waxes of component (b) to provide a solid cleansing composition with increased hardness whilst preventing cracking and surface oiling which can be caused by including such waxes in the solid cleansing composition. The combination of first and second emulsifiers suitably ensures that the carrier component (b) (which suitably includes an oil as defined above such as a triglyceride, and the one or more waxes) is fully emulsified in the composition throughout the manufacturing, melting and pouring stages which the composition is intended to go through. Suitably the excellent foaming and cleansing properties of the solid cleansing composition are maintained in this embodiment having increased hardness.

Suitably component (c) comprising the first emulsifier and the second emulsifier constitutes at least 4% wt and up to 14% wt of the solid cleansing composition, suitably from 6 to 12% wt or from 8 to 12% wt of the solid cleansing composition.

Suitably the first emulsifier provides from 1 to 5% wt of the solid cleansing composition, suitably from 2 to 4% wt of the solid cleansing composition.

Suitably the second emulsifier provides from 5 to 9% wt of the solid cleansing composition, suitably from 6 to 8% wt of the solid cleansing composition.

Suitably component (c) provides from 8 to 12% wt of the solid cleansing composition wherein the first emulsifier provides from 2 to 4% wt of the solid cleansing composition and the second emulsifier provides from 6 to 8% wt of the solid cleansing composition.

In such embodiments the solid cleansing composition suitably comprises:
(a) at least 12% wt of a non-soap anionic surfactant having a melting point of at least 45° C.;
(b) 16 to 28% wt of a carrier having a melting point of at least 45° C.;
(c) from 8 to 12% wt of an emulsifier;
(d) at least 8% wt of a humectant; and
(e) at least 8% wt water;
(f) a second emulsifier
   wherein the composition is a solid throughout the range from 0° C. to 40° C.;
   wherein component (b) comprises one or more waxes which provide from 2 to 8% wt of the solid cleansing composition and an oil, suitably a triglyceride or a hydrogenated triglyceride, which provides from 14 to 20% wt of the solid cleansing composition; and wherein component (c) comprises a first emulsifier having an HLB value of from 8 to 10 and a second emulsifier having an HLB value of from 3 to 6.

Throughout this specification “% wt” means percentage by weight of the composition which is then being defined.

The invention will now be further described by way of illustration with reference to the following examples.

### EXAMPLE 1

The following ingredients were employed in the amounts stated below.

| Ingredient | As added % wt |
|---|---|
| Water | 25.0 |
| PUREACT I-78 | 31.5 |
| Glycerol | 17.5 |
| Tetrasodium glutamate diacetate | 0.2 |
| Monopropyl glycol (MPG) | 2.0 |
| DUROSOFT PG4L-SG (emulsifier) | 7.8 |
| Hydrogenated sunflower oil | 16.0 |

PUREACT 1-78 is a Trade Mark of Innospec Performance Chemicals, of Ellesmere Port, UK. Its major component is sodium cocoyl isethionate (SCI), 83-89% wt; generally also having <1% wt water, <14% wt coconut fatty acid and 4-7% wt sodium isethionate.

DUROSOFT PG4L-SG (formerly known as DUROSOFT PK-SG) is a Trade Mark of Stephenson Group Limited of Leeds, UK. It is an ester of palm kernel oil and polyglycerol-4 (average Mw=250). DUROSOFT PG4L-SG has an HLB value of 8.8.

The emulsifier DUROSOFT and the glycerol were added to the water, which was at 90° C. The sodium cocoyl isethionate (SCI) was melted and blended to the composition. The hydrogenated sunflower oil was melted and was added last together with any further ingredients. The mass was worked for about 5 hours, whilst still liquid/molten. The resulting emulsion, a readily flowable liquid, was poured into a mould and allowed to cool under ambient conditions, producing a block of 11.5 kg weight.

The pH of the solid cleansing composition was 6-7 at 10% dilution, as measured by a commercial pH meter.

The moisture level of the cooled block was approximately 20 wt %; during the method some water was lost to the atmosphere. The solidified composition was as follows:

| Ingredient of solid composition | % wt |
|---|---|
| Water | 20.0 |
| PUREACT I-78 | 33.6 |
| Glycerol | 18.7 |
| Tetrasodium glutamate diacetate | 0.2 |
| Monopropyl glycol (MPG) | 2.1 |
| DUROSOFT PG4L-SG (emulsifier) | 8.3 |
| Hydrogenated sunflower oil | 17.1 |

The resulting block could be heated (for example to temperatures in the range 65° C. to 85° C.), thereby re-melting it, additised with any desired additive, for example selected from colorants, opacifiers and perfumes, and cast into small individual cleansing bars or tablets for personal use. It is found that such bars or tablets form perfectly without any degradation of the cleansing composition, and do not crack, shrink or distort in any way. The emulsification and humectant system is highly effective is stabilising the composition in the solid and liquid phases.

The cleansing bars can be used for skin and hair cleansing.

### EXAMPLE 2

The following ingredients were employed in the amounts stated below.

| | As added | In solid composition |
|---|---|---|
| Ingredient | % wt | % wt |
| Water | 25.0 | 20.0 |
| PUREACT I-78 | 20.8 | 22.2 |
| Glycerol | 27.0 | 28.8 |
| Tetrasodium glutamate diacetate | 0.2 | 0.2 |
| Monopropyl glycol (MPG) | 1.0 | 1.1 |
| DUROSOFT PG4L-SG (emulsifier) | 8.0 | 8.5 |
| Hydrogenated sunflower oil | 18.0 | 19.2 |

The method was carried out as described in Example 1. The composition of the resulting solid composition is as shown above. It will be seen that there was a small water loss.

As with Example 1, the physical properties, stability, re-melt performance and cleansing performance of the cleansing composition were excellent.

### EXAMPLE 3

The following ingredients were employed in the amounts stated. Addition of the ingredients was in the order stated, starting with water.

| | As added | In solid composition |
|---|---|---|
| Ingredient | % wt | % wt |
| Water | 24.5 | 19.5 |
| PUREACT I-78 | 23.6 | 25.2 |
| Glycerol | 25.0 | 26.7 |
| Tetrasodium glutamate diacetate | 0.2 | 0.2 |
| Monopropyl glycol (MPG) | 1.0 | 1.1 |
| DUROSOFT PG4L-SG (emulsifier) | 7.0 | 7.5 |
| Hydrogenated sunflower oil | 18.7 | 19.9 |

The method was carried out as described in Example 1. The composition of the resulting solid composition is as shown above. It will be seen that there was a small water loss.

As with Example 1, the physical properties, stability, re-melt performance and cleansing performance of the cleansing composition were excellent.

### EXAMPLE 4

The following ingredients were employed in the amounts stated. Addition of the ingredients was in the order stated, starting with water.

| | As added | In solid composition |
|---|---|---|
| Ingredient | % wt | % wt |
| Water | 25.0 | 20.0 |
| PUREACT I-78 | 20.0 | 21.3 |
| Glycerol | 26.0 | 27.7 |
| Tetrasodium glutamate diacetate | 0.2 | 0.2 |
| Monopropyl glycol (MPG) | 1.0 | 1.1 |
| DUROSOFT PG4L-SG (emulsifier) | 8.8 | 9.4 |
| Sodium benzoate | 0.5 | 0.5 |
| Potassium sorbate | 0.5 | 0.5 |
| Hydrogenated sunflower oil | 18.0 | 19.2 |

The method was carried out as described in Example 1. The composition of the resulting solid composition is as shown above. It will be seen that there was a small water loss.

As with Example 1, the physical properties, stability, re-melt performance and cleansing performance of the cleansing composition were excellent.

### EXAMPLE 5

Example composition 5 was prepared as described above for Example 1 with the following ingredients to produce a solid cleansing composition.

| | As added |
|---|---|
| Ingredient | % wt |
| Water | 24.90 |
| PUREACT I-78 | 24.60 |
| Glycerol | 16.30 |
| Tetrasodium iminodisuccinate | 0.20 |
| Monopropyl glycol (MPG) | 1.00 |
| DUROSOFT PG4L-SG (emulsifier) | 3.00 |
| Hydrogenated sunflower oil | 17.00 |
| DUROSOFT GML | 7.00 |
| Cetearyl Alcohol | 1.50 |
| Sunflower Wax | 4.50 |

DUROSOFT GML is Glyceryl Mono Laurate, an emulsifier having an HLB of 5.2.

As with Example 1, the physical properties, stability, re-melt performance and cleansing performance of the cleansing composition were excellent. The hardness of the cleansing composition was observed to be greater than the hardness of the cleansing composition of Examples 1-4 and no cracking or surface oiling was observed.

### EXAMPLE 6

Example composition 6 was prepared as described above for Example 1 with the following ingredients to produce a solid cleansing composition.

| | As added |
|---|---|
| Ingredient | % wt |
| Water | 22.20 |
| PUREACT I-78 | 24.60 |
| Glycerol | 19.00 |
| Tetrasodium iminodisuccinate | 0.20 |
| Monopropyl glycol (MPG) | 1.00 |
| DUROSOFT PG4L-SG (emulsifier) | 3.00 |
| Hydrogenated sunflower oil | 17.00 |
| DUROSOFT GMO | 7.00 |
| Cetearyl Alcohol | 1.50 |
| Sunflower Wax | 4.50 |

DUROSOFT GMO is Glyceryl Mono Oleate, an emulsifier having an HLB of 4.2.

As with Example 1, the physical properties, stability, re-melt performance and cleansing performance of the cleansing composition were excellent. The hardness of the cleansing composition was observed to be greater than the hardness of the cleansing composition of Examples 1-4 and no cracking or surface oiling was observed.

1. A solid cleansing composition, the composition comprising:
(a) at least 12% wt of a non-soap anionic surfactant having a melting point of at east 45° C.;
(b) at least 8% wt of a carrier having a melting point of at least 45° C.;
(c) at least 3% wt of an emulsifier;
(d) at least 8% wt of a humectant; and
(e) at least 8% wt water;
wherein the composition is a solid throughout the range from 0° C. to 40° C.

2. The solid cleansing composition as claimed in claim 1 wherein the composition may be melted at a temperature above 45° C. and re-solidified at a temperature of 45° C. or below.

3. The solid cleansing composition as claimed in claim 1 wherein the solid cleansing composition does not contain soap or starch.

4. The solid cleansing composition as claimed in claim 1 wherein the non-soap anionic surfactant comprises a sulfate, sulfonate, amphoacetate, sulfoacetate, sulfosuccinate, phosphate or carboxylate non-soap anionic surfactant; selected in each case to have a melting point above 45° C.

5. The solid cleansing composition as claimed in claim 4 wherein the non-soap anionic surfactant comprises an acyl isethionate.

6. The solid cleansing composition as claimed in claim 1 wherein the non-soap anionic surfactant constitutes at least 15% wt and up to 40% wt of the solid cleansing composition.

7. The solid cleansing composition as claimed in claim 1 wherein the carrier comprises one or more of a wax, a triglyceride, a fatty add or a fatty alcohol.

8.-9. (canceled)

10. The solid cleansing composition as claimed in claim 1 wherein the carrier constitutes at least 10% wt and up to 32% wt of the solid cleansing composition

11. The solid cleansing composition as claimed in claim 1 wherein the emulsifier has an HLB value of at least 6 and up to 12; and preferably at least 8 and up to 10.

12. The solid cleansing composition as claimed in claim 1 wherein the emulsifier is selected from sorbitan fatty acid esters, ethoxylated sorbitan fatty acid esters and glycerol or polyglycerol esters of fatty acids.

13. (canceled)

14. The solid cleansing composition as claimed in claim 1 wherein the emulsifier constitutes at least 4% wt and up to 14% wt of the solid cleansing composition.

15. The solid cleansing composition as claimed in claim 1 wherein the humectant is a humectant polyhydric alcohol or a humectant ester or humectant ether thereof.

16. The solid cleansing composition as claimed in claim 15 wherein the humectant is selected from compounds having from 2 to 6 hydroxyl groups and their di-, tri-, and oligomeric forms.

17. (canceled)

18. The solid cleansing composition as claimed in claim 1 wherein water constitutes at least 12% wt and up to 40% wt of the solid cleansing composition.

19. The solid cleansing composition as claimed in claim 1 which comprises:
(a) at least 15% wt and up to 40% wt of a non-soap anionic surfactant having a melting point of at least 45° C.;
(b) at least 10% wt and up to 32% wt of a carrier having a melting point of at least 45° C.;
(c) at least 4% wt and up to 14% wt of an emulsifier;
(d) at least 12% wt and up to 40% wt of a humectant;
(e) at least 10% wt and up to 36% wt of water;
wherein components (a) to (e) make up at least 70% and preferably at least 85% of the weight of the solid cleansing composition.

20.-22. (canceled)

23. The solid cleansing solid cleansing composition as claimed in claim 1 which has a completely or predominantly homogeneous microstructure.

24. (canceled)

25. An emulsion for the preparation of a solid cleansing composition, the emulsion comprising
(a) at least 11% wt of a non-soap anionic surfactant having a melting point of at least 45° C.;
(b) at least 7% wt of a carrier having a melting point of at least 45° C.;
(c) at least 3% wt of an emulsifier;
(d) at least 7 wt % of a humectant; and
(e) at least 10% wt water;
wherein the emulsion is flowable at a temperature of at least 45° C.

26. An emulsion for the preparation of a solid cleansing composition, the emulsion comprising
(a) at least 14% wt and up to 36% wt of a non-soap anionic surfactant having a melting point of at least 45° C. (and preferably at least 16% wt and up to 28% wt of this component);
(b) at least 10% wt and up to 32% wt of a carrier having a melting point of at least 45° C. (and preferably at least 12% wt and up to 24% wt of this component);
(c) at least 4% wt and up to 14% wt of an emulsifier (and preferably at least 6% wt and up to 12% wt of this component);
(d) at least 10 wt % and up to 36% wt of a humectant (and preferably at least 12% wt and up to 30% wt of this component); and
(e) and at least 12% wt and up to 40% wt water (and preferably at least 14% wt and up to 32% wt of this component);
wherein the emulsion is flowable at a temperature of at least 45° C., and
wherein components (a) to (e) make up at least 70%, preferably at least 80%, more preferably at least 90% of the weight of the emulsion.

27. A method of making a solid cleansing product as claimed in claim 1, the method comprising:
(i) providing the emulsion at a temperature at which it is flowable, and
(ii) subjecting the emulsion to cooling and solidification to form the solid cleansing product;
wherein the method takes place without water loss or with water loss not exceeding 50% of the mass of water added to form the emulsion; preferably not exceeding 40% of the mass of water added to form the emulsion; and preferably not exceeding 30% of the mass of water added to form the emulsion.

28. A method of providing individual personal cleansing bars by re-melting a larger solid cleansing product as claimed in claim 1, adding any colorants, perfumes that are required and casting the flowable composition into moulds.
